(19) United States
(12) Patent Application Publication
(10) Pub. No.: US 2003/0133899 A1
(43) Pub. Date: Jul. 17, 2003

(54) Personal cleansing compositions that contain surfactants, co-surfactants, water insoluble solids and/or liquids and cationic conditioning polymers

(75) Inventors: Shimei Fan, Inverness, IL (US); Teresa Jolanta Dowell, Downers Grove, IL (US); Tirucherai Varahan Vasudevan, Lake Zurich, IL (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/320,112

(22) Filed: Dec. 16, 2002

Related U.S. Application Data
(63) Continuation-in-part of application No. 10/001,558, filed on Oct. 24, 2001.
(60) Provisional application No. 60/244,448, filed on Oct. 31, 2000.

(51) Int. Cl. A61K 7/06; A61K 7/11; A61K 7/075; A61K 7/08

(52) U.S. Cl. 424/70.17; 424/70.22; 424/70.31; 424/70.21

(57) ABSTRACT

A cleansing and conditioning cosmetic composition containing a mixture of anionic, nonionic or amphoteric surfactants or mixtures thereof, with a cosurfactant that is alkoxylated nonionic surfactant, a water insoluble component as defined herein and a cationic conditioning polymer. The composition provides excellent cleansing and conditioning for hair/ skin and produces a copious, creamy lather.

### BACKGROUND OF THE INVENTION

Many cosmetic compositions are also cleansing compositions and thus have a surfactant base. Thus, there is a need for surfactant based systems from which cosmetically active ingredients can be efficiently deposited onto skin or hair. Many current surfactant based systems do not allow for such deposition.

There is also a need for this deposition across a whole range of cosmetic compositions which include hair shampoos, hair conditioners, sunscreens, deodorants, antiperspirants, insect repellants, lipsticks, lip balms, mousses, skin moisturizing compositions, anti-wrinkling compositions, antibacterial compositions, anti-fungal compositions, topical anesthetics; skin rash, skin disease, and dermatitis medications; anti-itch compositions, acne treatment preparations, burn relief medications, sunburn relief medications; medications for the relief of seborrhea, psoriasis, and dandruff; skin cleansing compositions, and compositions for relief from insect bites.

The present invention provides such surfactant bases and cosmetic compositions.

Publications, which relate to the invention, are as follows:

WO 9517880 discloses mild, high lathering shampoo compositions with high deposition of functional materials, the shampoo compositions comprising (a) from about 5% to about 40% by weight surfactant system (i) from 80% to 99% by weight of the surfactant system, anionic surfactants which are alkyl ethoxylated sulfates and alkyl sulfates in a ratio between about 1:1 to about 1:0 and (ii) about 1% to about 20% by weight of the surfactant system, polyhydroxy fatty acid amide surfactants; 0.05% functional materials; 35% to about 95% water;

WO 9217154 discloses hair conditioning compositions containing a detersive surfactant component, a silicone hair conditioning agent, water and preferably a suspending agent for the silicone conditioning agent. The detersive surfactant component comprises at least in part polyethylene glycol/glycerol fatty ester nonionic surfactant.

RU 2129860 C1 discloses a shading shampoo which contains surface active substances including nonionizing and ionogenic substances, anionic substances, softening components, color, flavor, water. Nonionizing substances additionally have ethoxymonoethanolamide of synthetic fat acids of fraction C10—C16 -syntamide-5. Glycerine or carbamine is used as a softening component. Shampoo additionally has carboxymethylcellulose and formalin. The components are taken in determined ratio.

### SUMMARY OF THE INVENTION

The present invention relates to rinse-off cleansing compositions, especially cleansing compositions containing conditioning oils and cationic conditioning polymers. Examples of such compositions are conditioning shampoos for cleaning and conditioning hair, and body washes for cleaning and conditioning skin.

When washing hair or skin with conventional, non-conditioning cleansing compositions the natural oils are removed together with unwanted oils and dirt. When excessive amounts of natural oils are removed, especially due to frequent washing, the hair or skin becomes dry. Such dryness causes hair to become raspy, less easy to comb and to build-up static that results in “flyaway”. In the case of skin, the dryness results in enhanced removal of moisture and, subsequently, cracking of the skin.

Hair and skin conditioners have been developed in order to restore the condition of the hair or skin, from the damage caused by washing, to its pre-washed or normal state. However, use of such products involves expensive and time-consuming additional steps.

Non-volatile silicone conditioning oils can be efficiently deposited onto skin and hair by directly incorporating them into the cleansing composition which is generally a mixture of anionic and nonionic/ amphoteric detersive surfactants. This process usually results in production of dispersions of silicone oils with a particle sizes of greater than 2 μm. Such large particles of silicone and hydrocarbon oils have a detrimental influence on the lathering characteristics of the cleansing composition due to ‘anti-foam’ effect.

Incorporation of silicone oil as a preformed aqueous emulsion into the cleansing composition enables attainment of particle size of less than 2 μm that has minimal effect on the lathering characteristics of the cleansing composition. Incorporation of silicone oils as a preformed emulsion also renders manufacturing of the compositions easier than when they are directly added into the composition. However, the efficiency of deposition on hair or skin of such small particles of silicone or hydrocarbon oils is generally very poor and the cleansing compositions containing dispersions of such small particles of silicone or hydrocarbon oils provide minimal or no conditioning.

Reid and Murray (U.S. Pat. No. 5,085,857) have disclosed cleansing compositions comprising a combination of a particular type of cationic conditioning polymer (guar hydroxypropyl trimethyl ammonium chloride) and an aqueous emulsion of non-volatile silicone oils of particle size less than 2 μm. These compositions impart improved conditioning benefit to hair with none of the undesirable dulling effects or greasy build-up seen with other conditioning products, and without a need for an expensive and inconvenient two-step washing and conditioning process. These compositions produce a creamy, copious lather.

We, unexpectedly, have found that by using a specific type of nonionic surfactant, namely alkoxylated nonionic surfactants, along with anionic surfactants, cationic polymer and silicone emulsions, high levels of silicone deposition, and hence conditioning, can be achieved. Additionally, we found that the efficiency of silicone deposition goes through a maximum at an optimum level of alkoxylation of the surfactant mixture. The optimum number of alkoxylation may depend on the specific surfactant mixture.

Compositions of the invention may comprise a mixture of:

a) from about 2 to about 40% by weight of a surfactant selected from the group consisting of anionic, amphoteric, non-ionic surfactants, and mixtures thereof;

b) from about 0.01 to about 20% by weight of a co-surfactant which is an alkoxylated nonionic surfactant;

c) from about 0.001 to about 10% by weight of a cationic polymer;

d) from about 0.01 to about 30% by weight of water insoluble components with an average particle size of less than about 2μm; and

e) from 0.001 to 10% by weight of an anti-dandruff agent.

The invention also comprises a method of treating the hair and/or skin which involves contacting the hair and/or skin with a composition of the invention.

### DETAILED DESCRIPTION OF THE INVENTION

As used herein % means weight % of the total composition unless otherwise specified. Degrees are in degrees Celsius unless otherwise specified. As used herein “turbid” means that a dilution of a premixture of a, b, and c above, (that is, in the absence of d)—‘water insoluble components’) with water at a ratio of premixture: water at about 1:10 measures at least about 3 NTU using the following protocol.

Turbidity protocol:

a) Background turbidity (Tb)

Fill the sample cell (Hach reference: 20849-00) with said premixture in the absence of ‘water insoluble components’ and leave it standing for 24 hours to release any trapped air. Turbidity (NTU) is then measured on a Hach RATIO/XR with the measurement being taken at 5 minutes after inserting the sample cell into the turbidity meter.

b) Dilution turbidity (Td)

3 g of said premixture above, in the absence of ‘water insoluble components’, and 27 g of soft water are added in a sample cell (Hach reference: 20849-00). The mixture is immediately mixed vigorously using a magnetic stirrer for 30 seconds and then sonicated for 1 minute (Sonicator used: BRANSON 1210). The sample cell is inserted into a turbidity meter (Hach RATIO/XR turbidmeter) immediately and the dilution turbidity measurement, Td (NTU) is taken at 5 minutes after insertion of the sample cell into the Hach RATIO/XR.

c) The turbidity value (in NTU units) quoted in this application is calculated as Td-1/10 Tb.

Another embodiment of the invention is a composition comprising a mixture of:

a) from about 2 to about 40% by weight of a surfactant selected from the group consisting of anionic, amphoteric, non-ionic surfactants, and mixtures thereof;

b) from about 0.01 to about 20% by weight of a co-surfactant which is alkoxylated nonionic surfactant, the alkoxylated nonionic surfactant not being an ethoxylated fatty alcohol containing 6 to 30 ethylene oxide groups;

c) from about 0.001 to about 10% by weight of a cationic polymer;

d) from about 0.01 to about 30% by weight of water insoluble components with an average particle size of less than about 2μm; and

e) a water soluble or insoluble anti-dandruff agent

More preferably, compositions of the invention comprise a mixture of:

a) from about 5 to about 25% by weight of surfactant selected from the group consisting of anionic, amphoteric, non-ionic surfactants, and mixtures thereof;

b) from about 0.05 to about 10% by weight of a co-surfactant which is alkoxylated nonionic surfactant;

c) from about 0.01 to about 1% by weight of a cationic polymer;

d) from about 0.05 to about 10% by weight of water insoluble components with an average particle size of less than 2 μm; and

e) a water soluble or insoluble anti-dandruff agent

What follows is a description of the ingredients which may be used in the compositions of the invention.

Surfactants

The compositions according to the invention comprise a surfactant selected from the group consisting of anionic, nonionic, amphoteric surfactants, and mixtures thereof.

Nonlimiting, suitable anionic surfactants are the alkyl sulfates, alkyl ether sulfates, alkaryl sulfonates, alkaryl isethionates, alkyl succinate, alkyl sulfosuccinates, N-alkoyl sarcosinates, alkyl phosphates, alkyl ether phosphates, alkyl ether carboxylates, and alpha-olefin sulfonates, especially their sodium, magnesium, ammonium and mono-, di- and triethanolamine salts. The alkyl and acyl groups generally contain from 8 to 18 carbon atoms and may be saturated and/or unsaturated. The alkyl ether sulfates, alkyl ether phosphates and alkyl ether carboxylates may contain from 1 to 10 ethylene oxide or propylene oxide units per molecule, and preferably contain 1 to 3 ethylene oxide units per molecule. Other nonlimiting and suitable anionic surfactants include sodium oleyl succinate, ammonium lauryl sulfosuccinate, ammonium lauryl sulfate, sodium dodecylbenzene sulfonate, triethanolamine dodecylbenzene sulfonate, sodium cocoyl isethionate, sodium lauroyl isethionate and sodium N-lauryl sarcosinate. The most preferred anionic surfactants are sodium lauryl sulfate [SLS], ammonium lauryl sulfate [ALS], sodium lauryl ether sulfate with 1 EO, 2EO and 3EO [SL(EO)₁₋₃S] and ammonium lauryl ether sulfate with 1 EO, 2EO and 3EO [AL(EO)₁₋₃S].

The nonionic surfactants suitable for use in the compositions of the invention may have less than about 2 EO and may include condensation products of aliphatic (C₈—C₁₈) primary or secondary linear or branched chain alcohols, phenols, esters, acids and amines. Other suitable nonionics include mono or dialkyl alkanolamides or alkyl polyglucosides. Nonlimiting examples of nonionic surfactants include coco mono or diethanolamide, coco mono isopropanolamide, and coco di glucoside.

The amphoteric surfactants suitable for use in the compositions of the invention may include alkyl amine oxides, alkyl betaines, alkyl amidopropyl betaines, alkyl sulfobetaines, alkyl glycinates, alkyl carboxy glycinates, alkyl ampho propionates, alkyl amidopropyl hydroxysultaines, acyl taurates and acyl glutamates wherein the alkyl and the acyl groups have from 8 to 18 carbon atoms. Examples include lauryl amine oxide, cocodimethyl sulfopropyl betaine and preferably lauryl betaine, cocamidopropyl betaine (CAPB) and sodium cocamphopropionate.

The surfactants may be present in the range from about 2 to about 40% by weight, preferably about 5 to about 30% by weight and more preferably from about 8 to about 20% by weight and most preferably from about 10 to about 15% by weight.

Co-surfactants

For the purpose of this invention, alkoxylated nonionic surfactants, which are a class of nonionic surfactants, are defined specifically as co-surfactants. Again, for the purpose of this invention only, alkoxylated nonionic surfactants, which are co-surfactants, are differentiated from the other nonionic surfactants defined previously, in that alkoxylated nonionic surfactants, which are co-surfactants, contain at least 2 or more alkylene oxide groups. (By contrast, nonionic surfactants with EO less than 2 are considered to be surfactants as described in the paragraphs above.) Examples of alkoxylated nonionic surfactants include, but are not limited to, condensation products of linear or branched fatty chain alcohols, acids, phenols, esters, glycerides, amines and amides. The preferred co-surfactants are ethoxylated nonionic surfactants with ethylene oxide (EO) groups in the range of 2 -12 and most preferably from 2 to 6. Especially preferred nonionic surfactants are ethoxylated fatty amides with the (EO) groups in the range of 2-12, most preferably 2 to 6.

The co-surfactants are present preferably in the range of 0.05 to 20% by weight, more preferably from 0.1 to 10% by weight and most preferably from 0.2 to 5% by weight.

Cationic polymer

Nonlimiting, cationic polymers that can be used in compositions of the invention include cationic cellulose derivatives, cationic starches, copolymers of a dialkyl quaternary ammonium salt and acrylamide, quaternized polyvinylpyrrolidone, quaternized vinylpyrrolidone vinylimidazol polymers, polyglycol amide condensates, quaternized collagen polypeptide, polethylene amine, cationized silicon polymer, cationic silicone polymers, copolymers of adipic acid and dimethylamino- hydroxypropyl diethylene triamine, polyaminopolyamide and their water soluble crosslinked polymers, cationic chitin derivatives, and cationic guar gums. The preferred cationic conditioning polymer is a cationic derivative of guar gum. The most preferred cationic polymer is guar hydroxy propyl trimethyl ammonium chloride.

The cationic polymers are preferably present in the range from 0.001 to 10% by weight, more preferably from 0.01 to 1% by weight and most preferably from 0.05 to 0.5% by weight.

Anti-dandruff/anti-fungal agents

Anti-dandruff/anti-fungal agents may be either soluble or insoluble in the compositions of the invention depending on the type and concentration of these agents. Nonlimiting examples of anti-dandruff/anti-fungal agents that can be used in the compositions of the invention are zinc pyrithione, climbazole, ketoconazole and octopirox. Anti-dandruff agents can be used alone or in mixtures of one or more such agents. Anti-dandruff agents typically display a minimum inhibitory concentration of about 50 mg/ml or less against Malassezia.

The anti-dandruff/antifungal agent may be present in the composition in an amount of from 0.001% to 10% by weight, preferably from 0.1 % to 5% by weight, more preferably from 0.5% to 3% by weight.

Water insoluble components

Water insoluble components that can be used in compositions of the invention include, but are not limited to, particulate materials such as oil droplets, silica and polymeric latex particles. The oil phase can comprise a volatile oil phase, a nonvolatile oil phase or a mixture thereof. The volatile oil phase can comprise a volatile silicone compound, a volatile hydrocarbon compound or a mixture thereof. Preferably, the volatile oil phase comprises a volatile silicone compound. Exemplary volatile compounds are listed in U.S. Pat. No. 5,589,177 which is hereby incorporated by reference.

The cyclic, low molecular weight, volatile polydimethylsiloxanes, designated in the CTFA International Cosmetic Ingredient Dictionary, 4th Ed., Cosmetic, Toiletry and Fragrance Association, Washington, D.C. (1991) (hereinafter CTFA Dictionary which is hereby incorporated by reference). as cyclomethicones, are the preferred siloxanes used in compositions of the present invention and are listed in U.S. Pat. No. 5,589,177.

The volatile oil phase also can comprise a volatile hydrocarbon compound. Volatile hydrocarbon compounds are listed in U.S. Pat. No. 5,589,177. The volatile hydrocarbon compounds perform the same function and provide essentially the same benefits as the volatile silicone compounds.

As previously stated, the oil phase also can be a nonvolatile oil phase. The nonvolatile oil phase comprises a nonvolatile silicone compound, a nonvolatile hydrocarbon, or a mixture thereof. Preferably, the nonvolatile oil phase comprises a nonvolatile silicone compound. The nonvolatile oil phase does not evaporate from the skin or hair. The nonvolatile oil phase boils at atmospheric pressure at a temperature above about 250 degree(s) C. Exemplary nonvolatile silicone compounds are listed in U.S. Pat. No. 5,085,857 which is incorporated herein by reference.

The nonvolatile oil phase can also comprise a nonvolatile hydrocarbon compound, such as mineral oil. Other exemplary nonvolatile hydrocarbon compounds that can be incorporated into the oil phase include, but are not limited to, a branched 1-decene oligomer, like 1-decene dimer or a polydecene. The oil phase also optionally can comprise (1) an oil, such as jojoba oil, wheat germ oil or purcellin oil; or (2) a water-insoluble emollient, such as, for example, an ester having at least about 10 carbon atoms, and preferably about 10 to about 32 carbon atoms. Suitable esters are listed in U.S. Pat. No. 5,589,177.

Water insoluble components can also include materials such as hair fixatives, hydrophilic particles, conditioning materials, emulsion particles, wax particles, encaps, dye/color particles, anti-dandruff particles and mixtures thereof.

Water insoluble components can further include materials used in sunscreens, deodorants, antiperspirants, insect repellants, lipsticks, lip balms, mousses, skin moisturizing compositions, anti-wrinkling /anti-aging compositions, antibacterial compositions, antifungal compositions, topical anesthetics, skin rash, skin disease, and dermatitis medications, anti-itch compositions, acne treatment preparations, burn relief medications, sunburn relief medications, medications for the relief of seborrhea, psoriasis, and dandruff, skin cleansing compositions, and compositions for relief from insect bites.

For the purpose of this invention, water insoluble components are defined as materials, which have solubility in water not greater than about 0.01 g/l when measured at about 25° C. in a pH range between about 3.5 and about 8.

Water insoluble particulates are present in the range from about 0.01 to about 30% by weight, preferably from about 0.05 to about 20% by weight, more preferably from about 0.1 to about 10% by weight and most preferably from about 0.5 to about 5% by weight.

Optional Ingredients

Optional ingredients that can be used in compositions of the invention are now described.

Optional ingredients can include any ingredients which are customarily included in cosmetic products and which do not interfere with the deposition properties of the surfactant system.

As optional components for inclusion in the compositions of the invention, the following may be mentioned: pH adjusting agents, suspending agents and viscosity modifiers such as xanthan gum and cross-linked polycarboxylates, cosmetic fillers such as talc, kaolin, preservatives, coloring agents, dyes, proteins, herb and plant extracts, polyols and moisturizing ingredients such as glycerine. Benefit agents that can be incorporated include, but are not limited to, sunscreens and alpha hydroxy acids.

Compositions of the invention can be made by using starting materials that are known in the art or by using starting materials that are obtainable from materials known in the art.

The following examples are provided for the purpose of illustration only, and do not, in any way, limit the scope of the invention.

### EXAMPLE 1 (HAIR CARE COMPOSITIONS)

| Ingredient | wt % | | | | | | | | | |
|---|---|---|---|---|---|---|---|---|---|---|
| Formula # → | 1 | 2 | 3 | 4 | 5 | 6 | 7 | 8 | 9 | 10 |

| | | | | | | | | | | |
|---|---|---|---|---|---|---|---|---|---|---|
| ALS | 12.0 | 12.0 | 12.0 | 12.0 | | | | | | |
| AL(EO)₁S | | | | | 12.0 | 12.0 | 12.0 | | | |
| AL(EO)₂S | | | | | | | | 12.0 | 12.0 | 12.0 |
| CMEA | 1.80 | | | | 1.80 | | | 1.80 | | |
| CM(EO)₂A | | 1.80 | | | | 1.80 | | | 1.80 | |
| CM(EO)_{3.5}A | | | 1.80 | | | | | | | |
| CM(EO)_{4.5}A | | | | 1.80 | | | 1.80 | | | 1.80 |
| NH₄Cl | 1.00 | 1.00 | 1.00 | 1.00 | 1.00 | 1.00 | 1.00 | 1.00 | 1.00 | 1.00 |
| Jaguar C13S | 0.10 | 0.10 | 0.10 | 0.10 | 0.10 | 0.10 | 0.10 | 0.10 | 0.10 | 0.10 |
| DC 1784 | 2.00 | 2.00 | 2.00 | 2.00 | 2.00 | 2.00 | 2.00 | 2.00 | 2.00 | 2.00 |
| Propylene glycol | 0.50 | 0.50 | 0.50 | 0.50 | 0.50 | 0.50 | 0.50 | 0.50 | 0.50 | 0.50 |
| DI Water | to 100 | to 100 | to 100 | to 100 | to 100 | to 100 | to 100 | to 100 | to 100 | to 100 |
| Si on hair, ppm | 30 | 130 | 230 | 580 | 145 | 190 | 400 | 190 | 190 | 260 |

This example shows that for a given number of ethylene oxide groups in ammonium lauryl sulfate, an increase in the number of ethylene oxide groups in cocomonoethanolamide results generally in an increased silicone deposition.

| | |
|---|---|
| Jaguar C13S | Guar hydroxypropyl trimethyl ammonium chloride from |
| | Rhodia Inc., USA |
| DC 1784 | Dimethiconol emulsion from Dow Corning Inc., USA |
| CMEA | Cocomonoethanolamide |
| CM(EO)_{X}A | Ethoxylated cocomonoethanolamide |
| x | The number of ethylene oxide groups. |
| Superscript* | Silicone oil content measured as Si element by |
| | Inductively Coupled Plasma Spectrophotometer and are |
| | reported as micrograms dimethiconol per gram of hair. |

### EXAMPLE 2 (HAIR CARE COMPOSITIONS)

| Ingredient | wt % | |
|---|---|---|
| Formula # | 11 | 12 |
| Sodium lauryl sulfate, SLS | 12.0 | 12.0 |
| CMEA | 1.80 | |
| CM(EO)_{4.5}A | 1.80 | |
| Jaguar C13S | 0.10 | 0.10 |
| DC 1784 | 2.00 | 2.00 |
| Propylene glycol | 0.50 | 0.50 |
| Deionized water | to 100 | to 100 |
| Si on hair, ppm | 290 | 615 |

This example shows that in systems containing sodium lauryl sulfate and CM(EO)_{4.5}A results in significantly higher silicone deposition than when sodium lauryl sulfate and CMEA is used.

### EXAMPLE 3 (HAIR CARE COMPOSITIONS)

| Ingredient | wt % | | |
|---|---|---|---|
| Formula # | 13 | 14 | 15 |
| ALS | 6.0 | 6.0 | 6.0 |
| AL(EO)₁S | 6.0 | 6.0 | 6.0 |
| Ammonium chloride | 1.0 | 1.0 | 1.0 |
| CMEA | 1.8 | 1.2 | 0.9 |
| CM(EO)₅A | — | 0.6 | 0.9 |
| Jaguar C13S | 0.1 | 0.1 | 0.1 |
| DC 1784 | 2.0 | 2.0 | 2.0 |
| Propylene glycol | 0.5 | 0.5 | 0.5 |
| Carbapol (thickening agent) | 0.4 | 0.4 | 0.4 |
| Mica & titanium dioxide (pearling agent) | 0.1 | 0.1 | 0.1 |
| Other minors (Fragrance, preservatives) | 1.0 | 1.0 | 1.0 |
| Deionized water | to 100 | to 100 | to 100 |
| Si on hair, ppm | 30 | 260 | 560 |

This example shows that in systems containing mixtures of ammonium lauryl sulfate [ALS], ethoxylated ammonium lauryl sulfate [AL(EO)₁S], cocomonoethanolamide [CMEA] and ethoxylated cocomonoethanolamide [CM(EO)5A], an increase in [CM(EO)5A] content relative to cocomonoethanolamide results in an increase in silicone deposition.

### EXAMPLE 4 (HAIR CARE COMPOSITIONS)

| Ingredient | wt % | |
|---|---|---|
| Formula # | 16 | 17 |
| ALS | 6.0 | 6.0 |
| AL(EO)₁S | 6.0 | 6.0 |
| Ammonium chloride | 1.0 | 1.0 |
| CMEA | 1.8 | 1.2 |
| CM(EO)₅A | 0.6 | |
| Jaguar C13S | 0.1 | 0.1 |
| JJ 555 | 2.0 | 2.0 |
| Propylene glycol | 0.5 | 0.5 |
| Carbapol (thickening agent) | 0.4 | 0.4 |
| Ethylene glygol distearate (pearling agent) | 0.1 | 0.1 |
| Other minors (Fragrance, preservatives) | 1.0 | 1.0 |
| Deionized water | to 100 | to 100 |
| Si on hair, ppm | 260 | 445 |

Note: JJ 555 is a dimethiconol emulsion from GE Silicones, USA

This example shows that the general trend seen in Example 3 holds when a different dimethiconol emulsion other than DC1784 is used. The different dimethiconal emulsion used here is JJ 555.

On a laboratory bench scale, compositions of the invention are made by the following procedure:

For preparing a 1 kg batch, 100 grams of soft water are weighed into a beaker provided with an overhead mixer. Desired amounts of anionic and nonionic-co-surfactants are then slowly added and the contents well mixed. If the non-ionic co-surfactant is in a solid form at ambient temperature, the batch is then heated to about 70-80° C. to ensure a good mixing and then cooled back to ambient temperature. Any loss of water is then made-up. A 2% Carbapol solution was subsequently added and mixed well, and this is followed by the addition of cationic guar dispersion in propylene glycol (1:5 guar/PG). Non-volatile silicone emulsion, fragrances and other minor ingredients are then added to the mix. Salt solution and remainder of soft water are subsequently added to the contents.

### EXAMPLE 5

| Ingredient | wt % | | | |
|---|---|---|---|---|
| Formula# | 18 | 19 | 20 | 21 |
| ALS | 6.0 | 6.0 | 6.0 | 6.0 |
| AL(EO)₁S | 6.0 | 6.0 | 6.0 | 6.0 |
| CMEA | 1.20 | 1.20 | 1.20 | 1.20 |
| CM(EO)_{4.5}A | 0.60 | 0.60 | 0.60 | 0.60 |
| Jaguar Excel* | 0.10 | 0.10 | 0.10 | 0.10 |
| DC 2-1870** | 0.375 | 0.375 | 0.375 | 0.375 |
| Propylene glycol | 0.40 | 0.40 | 0.40 | 0.40 |
| Crinipan AD*** | 1.00 | 2.00 | 3.00 | 4.00 |
| Deionized water | to 100 | to 100 | to 100 | to 100 |
| Appearance | Clear | Clear | Clear | Cloudy |

*Jaguar Excel Guar hydroxypropyl trimethyl ammonium chloride supplied by Rhodia Inc., Cranbury, New Jersey USA
**DC 2-1870 Silicone microemulsion supplied by Dow Corning Inc., Midland, Michigan, USA supplied as 25% active and added at 1.5% level (0.375% active) to the composition described in Formulas 18, 19, 20 & 21
Crinipan AD antidandruff agent Climbazole, supplied by Haarmann & Reimer, Teterboro, New Jersey, USA

This example shows that antidandruff ingredient Climbazole is soluble up to between 3 to 4 wt % in the above mentioned shampoo compositions.

### EXAMPLE 6

| Ingredient | wt % | |
|---|---|---|
| Formula # | 22 | 23 |
| ALS | 6.0 | 6.0 |
| AL(EO)₁S | 6.0 | 6.0 |
| CMEA | 1.20 | 1.20 |
| CM(EO)_{4.5}A | 0.60 | 0.60 |
| Jaguar Excel* | 0.10 | 0.10 |
| DC 2-1870** | 0.375 | 0.375 |
| Propylene glycol | 0.40 | 0.40 |
| Octopirox*** | 10.00 | 10.60 |
| Deionized water | to 100 | to 100 |
| Appearance | Clear | Cloudy |

*Jaguar Excel Guar hydroxypropyl trimethyl ammonium chloride supplied by Rhodia Inc., Cranbury, New Jersey USA
**DC 2-1870 Silicone microemulsion supplied by Dow Corning Inc., Midland, Michigan, USA supplied as 25% active and added at 1.5% level (0.375% active) to the composition described in Formulas 18, 19, 20 & 21
Octopirox antidandruff agent supplied by Clariant Inc., Charlotte, North Carolina, USA

This example shows that antidandruff ingredient Climbazole is soluble up to 10 wt % in the above mentioned shampoo compositions.

### EXAMPLE 7

The following example of the invention can be made:

| Ingredient | wt % | |
|---|---|---|
| Formula # | 16 | 17 |
| ALS | 6.0 | 6.0 |
| AL(EO)₁S | 6.0 | 6.0 |
| Ammonium chloride | 1.0 | 1.0 |
| CMEA | 1.2 | 1.2 |
| CM(EO)₅A | 0.6 | 0.6 |
| Jaguar C13S | 0.1 | 0.1 |
| JJ 555 | 2.0 | 2.0 |
| Zinc pyrithione | 2.0 | 10.0 |
| Propylene glycol | 0.5 | 0.5 |
| Carbapol (thickening agent) | 0.4 | 0.4 |
| Ethylene glycol distearate (pearlizing agent) | 0.1 | 0.1 |
| Other minors (Fragrance, preservatives) | 1.0 | 1.0 |
| Deionized water | to 100 | to 100 |

### Method of Use of Compositions of the Invention

Compositions of the invention may be used as hair shampoos by applying said compositions to wet hair, lathering, then rinsing. Hair conditioners may be used after shampooing with compositions of the invention on an as-needed basis.

Compositions of the invention may be used as body washes by wetting the body in the shower, and then applying a composition of the invention, and then rinsing with water.

What is claimed is:

1. An aqueous rinse-off cleansing and conditioning composition comprising,
a) from about 2 to about 40% by weight of surfactant selected from the group consisting of an anionic surfactant, an amphoteric surfactant, a non-ionic surfactant, and mixtures thereof;
b) from about 0.01 to about 20% by weight of a co-surfactant which is an alkoxylated nonionic surfactant, the alkoxylated non-ionic surfactant not being an ethoxylated fatty alcohol containing 6 to 30 ethylene oxide groups;
c) from about 0.001 to about 10% by weight of a cationic polymer;
d) from about 0.01 to about 30% by weight of water insoluble components with an average particle size of less than 2 μm; and
e) from 0.001 to 10% by weight of a water soluble or insoluble anti-dandruff agent.

2. An aqueous rinse-off cleansing and conditioning composition comprising,
a) from about 2 to about 40% by weight of surfactant selected from the group consisting of an anionic surfactant, an amphoteric surfactant, a non-ionic surfactant, and mixtures thereof;
b) from about 0.01 to about 20% by weight of a co-surfactant which is an alkoxylated nonionic surfactant, the alkoxylated nonionic surfactant not being an ethoxylated fatty alcohol containing 6 to 30 ethylene oxide groups;
c) from about 0.001 to about 10% by weight of a cationic polymer;
d) from about 0.01 to about 30% by weight of water insoluble components with an average particle size of less than about 2 μm; and
e) from 0.001 to 10% by weight of a water soluble or insoluble anti-dandruff agent.
wherein a), b) and c) in a premixture with water at a ratio of premixture: water at about 1:10 forms a turbid mixture.

3. A composition according to claim 1 wherein said anionic surfactant is selected from the group consisting of the alkyl sulfates, alkyl ether sulfates, alkaryl sulfonates, alkaroyl isethionates, alkyl succinate, alkyl sulphosuccinates, N-alkoyl sarcosinates, alkyl phosphates, alkyl ether phosphates, alkyl ether carboxylates, alpha-olefin sulfonates and mixtures thereof.

4. A composition according to claim 1 wherein said amphoteric surfactant is selected from the group consisting of alkyl amine oxides, alkyl betaines, alkyl amidopropyl betaines, alkyl sulfobetaines, alkyl glycinates, alkyl carboxy glycinates, alkyl ampho propionates, alkyl amidopropyl hydroxysultaines, acyl taurates, acyl glutamates, and mixtures thereof.

5. A composition according to claim 1 wherein said amphoteric surfactant is acyl taurate, or acyl glutamate, and wherein said alkyl and acyl groups have from about 8 to about 18 carbon atoms.

6. A composition according to claim 1 wherein said amphoteric surfactant is an alkyl betaine selected from the group consisting of cocodimethyl sulfopropyl betaine, lauryl betaine, and mixtures thereof.

7. A composition according to claim 1 wherein said amphoteric surfactant is sodium amphopropionate.

8. A composition according to claim 1 wherein said nonionic surfactant is a nonionic surfactant having less than 2EO which is selected from the group consisting of condensation products of aliphatic (C₈—C₁₈) primary, secondary, linear, and branched fatty chain alcohols, acids, phenols, esters and amines; mono-, dialkyl alkanolamides; mono-, dialkyl polyglucosides; and mixtures thereof.

9. A composition according to claim 1 wherein the nonionic surfactant is selected from the group consisting of coco mono or diethanolamide; coco mono isopropanolamide; coco di glucoside; and mixtures thereof.

10. A composition according to claim 1 wherein the cosurtactant is selected from the group consisting of alkylene oxide condensation products of linear or branched fatty chain alcohols, acids, phenols, esters, glycerides, amines, amides; and mixtures thereof.

11. A composition according to claim 1 wherein the cosurfactant is selected from the group consisting of ethylene oxide condensation products of linear or branched fatty chain alcohols, acids, phenols, esters, glycerides, amines, amides; and
mixtures thereof.

12. A composition according to claim 1 wherein the cosurfactant is an ethoxylated cocomonoethanolamide with EO ranging from about 2 to about 12.

13. A composition according to claim 12 wherein the cosurfactant is an ethoxylated cocomonoethanolamide with EO ranging about 3 to about 6.

14. A composition according to claim 1 wherein the cationic conditioning polymer is selected from the group consisting of cationic cellulose derivatives, cationic starches, copolymers of a dialkyl quaternary ammonium salt and acrylamide; quaternized polyvinylpyrrolidone, quaternized vinylpyrrolidone vinylimidazol polymers, polyglycol amide condensates, quaternized collagen polypeptides, polyethylene amines, cationized silicon polymers, cationic silicone polymers, copolymers of adipic acid and dimethylaminohydroxypropyl diethylene triamine; polyaminopolyamide and their water soluble crosslinked polymers, cationic chitin derivatives, cationic guar gums, and mixtures thereof.

15. A composition according to claim 1 wherein the cationic conditioning polymer is guar hydroxypropyl trimethyl ammonium chloride.

16. A composition according to claim 1 wherein the water insoluble volatile liquid is an emulsion of cyclomethicone.

17. A composition according to claim 1 wherein the water insoluble non-volatile liquid is a silicone oil which is selected from the group consisting of polyalkyl siloxanes, polyalkyl aryl siloxanes and mixtures thereof.

18. A composition according to claim 16 wherein the water insoluble non-volatile liquid is a silicone oil which is selected from the group consisting of polydimethyl siloxane, polymethyl phenyl siloxane, and mixtures thereof.

19. A composition according to claim 1 wherein the water insoluble non-volatile liquid is a silicone oil that has an average particle size of from about 0.05 to about 2 μm.

20. A composition according to claim 1 which further comprises one or more components selected from the group consisting of pH adjusting agents, viscosity modifiers, talc, kaolin, suspending agents, preservatives, coloring agents, dyes, proteins, herb and plant extracts, polyols, alpha hydroxy acids and sunscreens.

21. A composition according to claim 1 wherein said surfactant and co-surfactant is a mixture comprising ALS/AL(EO)₁S/CMEA/CM(EO)₅A.

22. A composition according to claim 1 wherein said surfactant and co-surfactants is a mixture comprising ALS/AL(EO)₂S/CMEA/CM(EO)₅A.

23. A method for treating hair, which comprises contacting said hair with a composition according to claim 1.

24. A method for treating skin, which comprises contacting said skin with a composition according to claim 1.

25. A method for enhancing the deposition of a silicone on hair which comprises contacting said hair with an aqueous rinse-off cleansing and conditioning composition comprising:
a) from about 2 to about 40% by weight of surfactant selected from the group consisting of an anionic surfactant, an amphoteric surfactant, a nonionic surfactant, and mixtures thereof;
b) from about 0.01 to about 20% by weight of a co-surfactant which is an alkoxylated nonionic surfactant, the alkoxylated nonionic surfactant not being an ethoxylated fatty alcohol containing 6 to 30 ethylene oxide groups;
c) from about 0.001 to about 10% by weight of a cationic polymer;
d) from about 0.01 to about 30% by weight of water insoluble components with an average particle size of less than about 2 μm and wherein said water insoluble components comprise a silicone; and
e) from about 0.001 to about 10% by weight of a water soluble anti-dandruff agent.

26. A composition according to claim 1 wherein the anti-dandruff agent is zinc pyrithione.

27. A composition according to claim 26, wherein the zinc pyrithione is present at about 0.1 % by weight to about 10% by weight.

28. A composition according to claim 27 wherein the zinc pyrithione is present at about 0.5% by weight to about 3% by weight.

29. A composition according to claim 1, wherein the anti-dandruff agent is climbazole.

30. A composition according to claim 1 which is optically clear.

31. A composition according to claim 1 wherein the anti-dandruff agent is octopirox.

32. A composition according to claim 31 which comprises up to about 10% by weight of octopirox.

33. A composition according to claim 29 which comprises up to 3.5% by weight of climbazole.
